(19) United States
(12) Patent Application Publication
(10) Pub. No.: US 2004/0133193 A1
(43) Pub. Date: Jul. 8, 2004

(54) Methods and apparatus for treating intraluminal blockages

(76) Inventors: Jane P. Bearinger, Livermore, CA (US); Jeffrey A. Hubbell, Zurich (CH)

Correspondence Address: KENNETH J. MICHLITSCH, 822 SOUTH M STREET, LIVERMORE, CA, 94550, US

(21) Appl. No.: 10/641,983

(22) Filed: Aug. 15, 2003

Related U.S. Application Data
(60) Provisional application No. 60/403,901, filed on Aug. 16, 2002.

(51) Int. Cl. A61B 18/18

(52) U.S. Cl. 606/15

(57) ABSTRACT

The present invention provides methods and apparatus for treating intraluminal blockage using radical species generated with a photocatalyst. The photocatalyst may comprise, for example, a photocatalytic semiconductor, a photosensitizer, or a combination thereof. The radical species are brought into contact with the blockage, thereby locally oxidizing or transferring energy to the blockage, which disrupts the blockage. The photocatalyst is preferably disposed on the distal end of an optical fiber that is brought into close proximity or contact with the intraluminal blockage. The photocatalyst is then excited in a manner capable of generating radical species, for example, oxygen-containing radical species, in appropriate media.

### REFERENCE TO RELATED APPLICATION

The present application claims priority and the benefit of the filing date of provisional U.S. patent application Serial No. 60/403,901 filed Aug. 16, 2002, and takes advantage of that filing date.

### FIELD OF THE INVENTION

The present invention is related to localized treatment of intraluminal blockages. More particularly, this invention is related to methods and apparatus for disrupting thrombosis, blood clots, atherosclerotic plaque, stones, or other body lumen occlusions using radical species.

### BACKGROUND OF THE INVENTION

Stroke is defined as an acute loss of blood flow to regions of the brain. Most strokes are caused by a blood clot that blocks an artery feeding the brain. The loss of blood flow causes brain cells to die due to a lack of blood-borne oxygen and nutrients. Approximately 10 million people have strokes each year, and nearly 2 million of them die. As many as 15-30% of survivors suffer from permanent disability, and 20% may require long-term professional care. A key to effectively treating strokes is rapid intervention; if blood flow is restored within three to six hours, damage may be limited.

A variety of techniques have been proposed for treating blood clots and other vascular blockages, including a variety of localized, intravascular techniques utilizing catheters advanced to the vicinity of blockage. These include localized administration of TPA (a clot dissolving drug), high-pressure fluid jets, mechanical snares, Photodynamic Therapy (“PDT”) and photoacoustic emulsification.

Photodynamic Therapy (“PDT”) is a technique that may have utility in treating a variety of ailments by injuring targeted cell membranes via generation of highly energetic radical species with a photosensitive dye. One PDT procedure in clinical use today is the treatment of age-related macular degeneration. Typically, a photosensitive dye or drug is administered and allowed to locally accumulate over a period of time at a target site. Once a sufficient quantity of the photosensitive dye has accumulated, the target site is irradiated with incident light tuned to a specific wavelength that activates the photosensitive dye and generates the highly energetic radical species that cause injury to cells at the target site.

The radicals consist of singlet oxygen and other free radicals that are capable of damaging tumor cells and endothelial cells that line vasculature. The incident light used to generate radicals is normally applied with a non-thermal, low-intensity infrared laser. In addition to wavelength, laser parameters, such as fluence and irradiation time, must be adjusted for the specific clinical indication. Administration, dose, and localization of the photosensitizer must also be optimized.

Numerous photosensitive dyes are under investigation for PDT therapies. These include Benzoporphyrin, which is marketed under the trade name Visudyne by Novartis Opthalmics of Atlanta, Ga., and by QLT Therapeutics of Vancouver, British Columbia, Canada; Tin Ethyl Etiopurpurin, which is a lipophilic photosensitizer marketed as Purlytin by Miravant Medical Technologies of Santa Barbara, Calif., and by Pharmacia Opthalmics of Bridgewater, N.J.; lutetium texaphyrin, or LuTex, a hydrophilic synthetic molecule from Pharmacyclics of Sunnyvale, Calif.; NPe6 (mono-L-aspartyl chlorin e6); and ATX-S10.

PDT has several drawbacks. First, it is time-intensive. The photosensitive dye must be administered at the target site and allowed to accumulate before light activation may proceed. Second, it is cost-intensive. In addition to requiring a dedicated laser tuned to the peak absorption of the photosensitive dye, PDT procedures often require an infusion pump, as well as several dedicated personnel to assist in intravenous administration of the dye.

PDT is also complicated, requiring significant clinician training and creating a risk of error during clinical administration. Laser parameters, including fluence and irradiation time, must be optimized for the clinical indication. Dye administration parameters, including dose and localization, must also be optimized. A potential for migration of the dye into regions other than the target site is high. Furthermore, residual dye remains in the patient post-treatment, which often necessitates that the patient avoids sunlight for periods as long as 1 month post-procedure. Finally, to date, PDT has not been proven safe and effective for localized treatment of intravascular blockages, such as blood clots, thrombosis and other occlusions.

An additional technique for treatment of stroke is photoacoustic emulsification. Endovasix Corporation of Belmont, Calif., has developed a catheter with optical fibers that are coupled to a laser. The catheter is capable of generating acoustic energy in the form of pressure and shock waves for emulsifying clot material to very small particles. Localized heating of blood with a laser beam in the vicinity of a blood clot generates a vapor bubble that drives low-intensity, long-duration pressure waves. Additionally, the laser energy is deposited in the blood more rapidly than the blood can expand toward equilibrium, thereby forming short-duration, high-pressure shock waves. The concurrent pressure and shock waves are capable of emulsifying blood clots.

Photoacoustic emulsification has several drawbacks. First, production of low-pressure waves requires deposition of very high volumetric energy concentrations. High energy concentrations increase a risk of damage to vessel walls, especially in the tortuous blood vessels in the brain. Additionally, lasers operating at optimal wavelengths for energy deposition in blood are not readily available; techniques for producing such wavelengths may decrease reliability of the laser and/or add additional expense.

In view of the drawbacks associated with prior art techniques for treating intraluminal blockages, it would be desirable to provide methods and apparatus that overcome those drawbacks.

It would be desirable to provide methods and apparatus for treating intraluminal blockages that leave no foreign materials resident in the patient's body lumen post-treatment.

It would also be desirable to provide light-based methods and apparatus requiring relatively low energy concentrations.

It would be desirable to provide methods and apparatus for treating intraluminal blockages that do not require a concussive wave.

It would be desirable to provide light-based methods and apparatus that are faster, less expensive, simpler, and require less optimization of laser parameters by the clinician.

### SUMMARY OF THE INVENTION

In view of the foregoing, it is an object of the present invention to provide methods and apparatus for treating intraluminal blockages that overcome drawbacks associated with prior art techniques for treating intraluminal occlusions.

It is an object to provide methods and apparatus for treating intraluminal blockages that leave no foreign materials resident in the patient's body lumen post-treatment.

It is also an object to provide light-based methods and apparatus requiring relatively low energy concentrations.

It is an object to provide methods and apparatus for treating intraluminal blockages that do not require a concussive wave.

It is another object to provide light-based methods and apparatus that are faster, less expensive, simpler, and require less optimization of laser parameters by the clinician.

These and other objects of the present invention are accomplished by treating intraluminal blockages with radical species generated via a photocatalyst, such as a photocatalytic semiconductor, a photosensitizer, or a combination thereof, disposed on the distal end of an optical fiber. The radical species may be generated, for example, by coupling a proximal end of the optical fiber to an appropriate energy source, e.g. a laser, capable of exciting or forming electron hole pairs within the photocatalyst. Energy from the energy source is passed through the optical fiber to the photocatalyst, where it facilitates formation of the radical species in appropriate environments.

When the photocatalyst comprises a photocatalytic semiconductor, energy from the energy source generates electron hole pairs in the photocatalyst. The electron hole pairs generate radical species, such as oxygen-containing radical species, in appropriate environments. Preferred photocatalytic semiconductors include, but are not limited to, TiO₂, SnO₂, and an InTaO₄ compound doped with Ni. Preferred energy sources for use with photocatalytic semiconductors include, but are not limited to, UV and x-ray lasers.

When the photocatalyst comprises a photosensitizer, energy from the energy source excites the photosensitizer from a ground state to a singlet excited state. The singlet may decay to an intermediate triplet excited state, which is able to transfer energy to another triplet. Some molecules have a triplet ground state, for example, oxygen or O₂. Thus, energy may be transferred from the photosensitizer in the excited triplet state to the triplet ground state molecule, thereby exciting the molecule to a singlet state. A radical-generating reaction may then be achieved with the excited singlet state molecule, for example, a reaction generating oxygen-containing radical species. Molecules capable of forming radical species upon exposure to an excited photosensitizer will be apparent to those of skill in the art and preferably are provided at the distal end of the optical fiber, for example, thiohydroxamic esters. Unlike the liquid photosensitive dyes used in prior art Photodynamic Therapy (“PDT”) techniques, photosensitizers of the present invention are provided in solid form and/or are contained at the distal end of an optical fiber, thereby ensuring that the photosensitizer is not left within the patient post-treatment.

Preferred photosensitizers include, but are not limited to, photofrins, texaphyrins, metallotexaphyrins, porphyrins, hematoporphyrins, chlorins, bacteriochlorins, phthalocyanines and purpurins. Preferred energy sources for use with photosensitizers include, but are not limited to, visible light sources, such as light sources with wavelengths between about 550-850 nm, for example, visible laser light sources, such asHelium Neon (“HeNe”) lasers. Other light sources, such as UV light sources, will be apparent.

Radical species are brought into localized contact with an intraluminal blockage by disposing the distal end of the optical fiber in close proximity or contact with the blockage. The distal end of the optical fiber may be advanced proximate the blockage using, for example, well-known percutaneous techniques. When radicals are generated, they locally contact the blockage due to the location of the photocatalyst at the distal end of the optical fiber. The radical species oxidize or transfer energy to the blockage, which breaks up or dissolves the blockage.

It is expected that radical species generated at the photocatalyst will be transferred to the blockage along a substantially shortest distance path. Thus, only the blockage in close proximity to the photocatalyst will come into contact with the radical species. Portions of the patient's body lumen that are not contacted by the radical species are not expected to oxidize, dissolve, break up, etc. It should be noted that oxidation may be possible with excited singlet or triplet state molecules, in addition to radical species.

The region within the patient's body lumen in the vicinity of the blockage preferably comprises a medium capable of generating radical species in the presence of electron hole pairs or excited molecules, for example, an oxygen-containing medium, such as blood, water, oxygen, air, saline and combinations thereof. If an appropriate medium is not available at the target site, a clinical practitioner may provide it, for example, via a guiding or infusion catheter.

In a first embodiment of the present invention, a single optical fiber, proximally coupled to an appropriate energy source and having a photocatalyst at its distal end, is provided. In a second embodiment, a plurality of such optical fibers may be provided, either discretely or coupled. In a third embodiment, a plurality of coupled fibers is provided disposed about a central shaft. The central shaft optionally may have one or more lumens, such as a guide wire lumen and/or an infusion lumen for providing appropriate medium to the patient's body lumen in the vicinity of the blockage, such as a medium capable of generating radical species and/or a medium capable of cooling the patient's body lumen during treatment. Embolic protection devices and techniques may also be provided/employed.

A significant advantage of the present invention, as compared to prior art Photodynamic Therapy techniques, is that PDT requires introduction and local accumulation of a photosensitive dye or drug over a period of time at a target site. Such localized accumulation is difficult or impractical in many body lumens where fluids are flowing, such as in blood vessels containing blood. Conversely, the present invention only requires that the photocatalyst be exposed to an appropriate medium, which need not be localized nor allowed to locally accumulate over a period of time. The medium may be chosen such that a risk of harm to the patient due to the medium is negligible. Such media may include, for example, water, oxygen, air, saline and combinations thereof. Furthermore, the medium preferably is naturally occurring at the treatment site. For example, when treating an intravascular blockage, the medium may comprise blood. In such cases, no foreign material is left in the patient post-treatment, since the photocatalyst is disposed at the distal end of an optical fiber that is removed from the patient post-treatment.

As compared to prior art photoacoustic emulsification techniques, the present invention advantageously requires relatively low energy concentrations and does not require formation of a concussive wave.

Methods and apparatus for accomplishing the present invention are provided.

### BRIEF DESCRIPTION OF THE DRAWINGS

Further features of the invention, its nature and various advantages will be more apparent from the accompanying drawings and the following detailed description of the preferred embodiments, in which like reference numerals refer to like parts throughout, and in which:

FIGS. 1A-1C are schematic representations of theoretical photocatalyst reactions leading to generation of radical species: FIGS. 1A and 1B depict the formation of electron hole pairs in a photocatalytic semiconductor, while FIG. 1C depicts excitation of a photosensitizer;

FIGS. 2A and 2B are schematic representations of localized oxidation and/or energy transfer to an intraluminal blockage in the presence of radical species;

FIG. 3 is a schematic view of a first embodiment of apparatus of the present invention comprising a single optical fiber;

FIGS. 4A and 4B are schematic views of a second embodiment of apparatus of the present invention comprising a plurality of optical fibers;

FIGS. 5A-5C are schematic views of a third embodiment of apparatus of the present invention comprising a plurality of coupled optical fibers disposed about a central shaft; and

FIGS. 6A-6D are schematic views demonstrating a method of using the apparatus of FIG. 5C.

### DETAILED DESCRIPTION OF THE INVENTION

The present invention is related to localized treatment of intraluminal blockages. More particularly, the present invention is related to methods and apparatus for disrupting thrombosis, blood clots, atherosclerotic plaque, stones, or other body lumen occlusions using radical species.

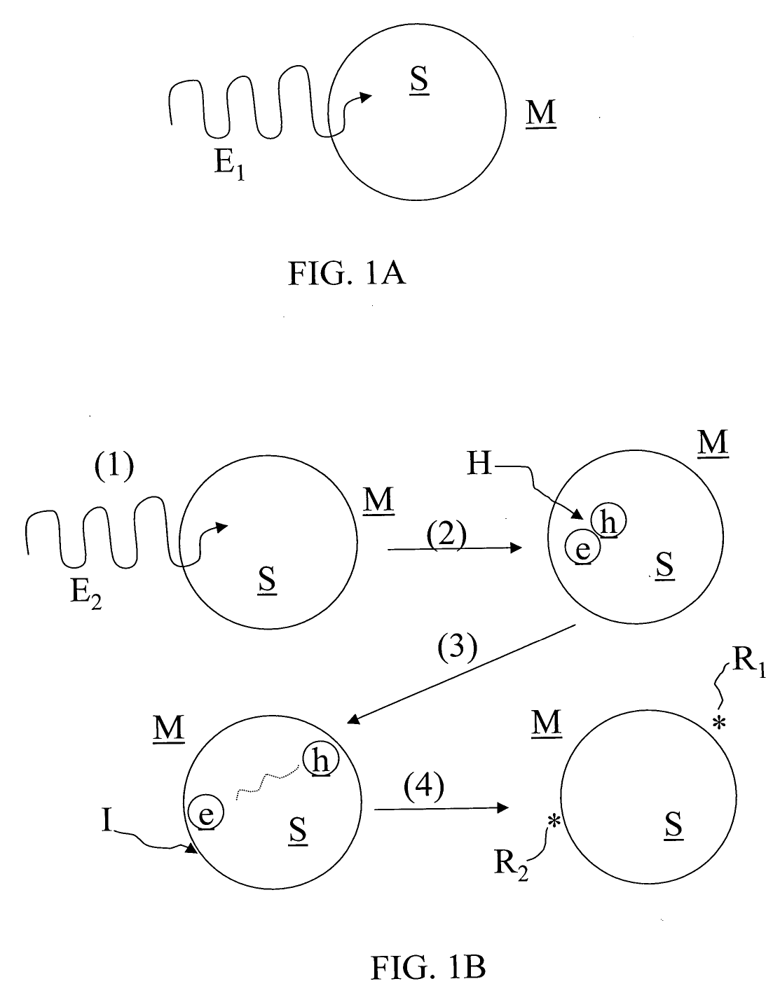

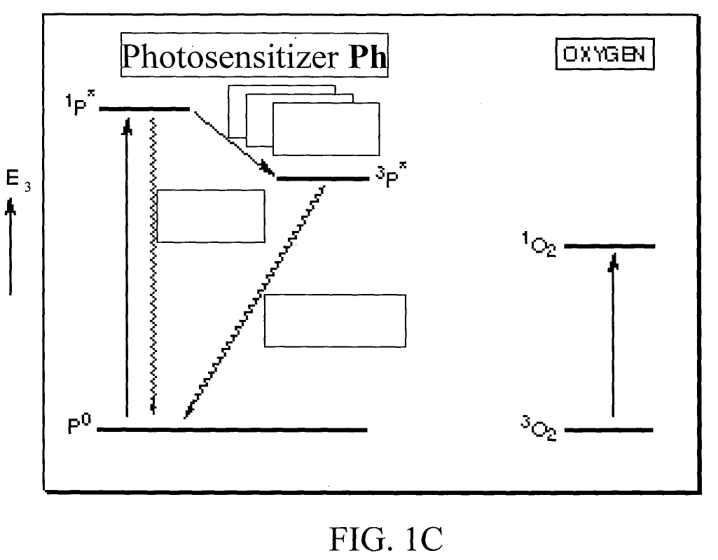

With reference to FIGS. 1 and 2, prior to discussion of apparatus and methods in accordance with the present invention, reactions encountered while practicing the present invention are described. Although these reactions are believed to be the mechanism by which the present invention may be practiced, the present invention is primarily concerned with the end result, i.e. treatment of intraluminal blockages. Thus, the reactions and purported mechanism are provided only for the benefit of the reader and should in no way be construed as limiting.

FIG. 1 describe photocatalyst reactions leading to generation of radical species. FIGS. 1A and 1B depict the formation of an electron hole pair in a photocatalytic semiconductor atom, with subsequent generation of radical species. FIG. 1C depicts excitation of a photosensitizer.

In FIG. 1A, photocatalytic semiconductor atom S is disposed in an oxygen-containing medium M, for example, H₂O, saline, air, or blood. Semiconductor atom S is contacted by energy quanta E₁ having an excitation energy below the band gap energy of semiconductor atom S. As an illustrative example, the band gap energy for photocatalytic semiconductor TiO₂ is about 3.2 eV. Since energy quanta E₁ has an excitation energy below the band gap of semiconductor atom S, the quanta does not generate an electron hole pair in semiconductor atom S.

In FIG. 1B, semiconductor atom S is contacted by energy quanta E₂ having an excitation energy above the band gap of semiconductor atom S. Energy quanta E₂ releases electron e and hole h within semiconductor S, which are collectively referred to as electron hole pair H. Electron hole pair H migrates to atom/medium interface I. Electron e and hole h interact with oxygen contained within medium M, thereby forming oxygen-containing radical species R₁ and R₂. R₁ is a hydroxyl radical, while R₂ is a super-anion oxide radical. Radical species R₁ and R₂ have cross-sections on the order of Angstroms or smaller. After a brief period, electron hole pairs that don't form radical species recombine.

For the exemplary embodiment of a TiO₂ photocatalytic semiconductor atom S exposed to energy quanta E₂ from a UV energy source, while immersed in fluid medium M comprising H₂O, the equations governing generation of radical species are as follows:

TiO₂+UV→e+h (1)

h+OH—→*OH (2)

e+O₂→O₂*— (3)

O₂*−+H₂O→HO₂*+OH— (4)

where ‘*’ denotes a radical species. This provides an overall reaction via TiO₂ catalysis of:

UV+O₂+H₂O→HO₂*+*OH (5)

Although FIGS. 1A and 1B are described with respect to an oxygen-containing medium, other mediums containing other elements capable of generating radical species in the presence of electron hole pairs will be apparent to those of skill in the art. One such medium is a nitrogen-containing medium. Others include reagents that may react across an unsaturated bond via a Michael-type addition mechanism.

Referring now to FIG. 1C, photosensitizer Ph is excited from ground state P⁰ to excited singlet state ¹p* by energy quanta E₃. Photosensitizer Ph decays from singlet state ¹p* to intermediate excited triplet state ³p*. While disposed in the triplet state, photosensitizer Ph is able to transfer energy to another triplet state molecule. Some molecules have a triplet ground state, for example, oxygen O₂, which is used in the exemplary embodiment of FIG. 1C.

As seen in FIG. 1C, energy is transferred from excited triplet state photosensitizer ³p* Ph to triplet ground state oxygen molecule ³O₂, thereby exciting the ³O₂ molecule to an excited singlet state ¹O₂. A radical-generating reaction may then be achieved with the excited singlet state molecule ¹O₂, for example, a reaction that generates oxygen-containing radical species. Molecules capable of forming radical species upon exposure to an excited photosensitizer will be apparent to those of skill in the art, for example, thiohydroxamic esters.

With reference to FIG. 2, treatment of an intraluminal blockage with radical species is described. It should be noted that treatment, e.g. oxidation, may be possible with excited singlet or triplet state molecules, in addition to radical species. Such treatment falls within the scope of the present invention.

In FIG. 2A, blockage B is bombarded by radical species R. Radical species R transfer energy and/or locally oxidize blockage B where the radical species contact the blockage, thereby breaking up or dissolving the blockage into smaller emboli Em, as seen in FIG. 2B.

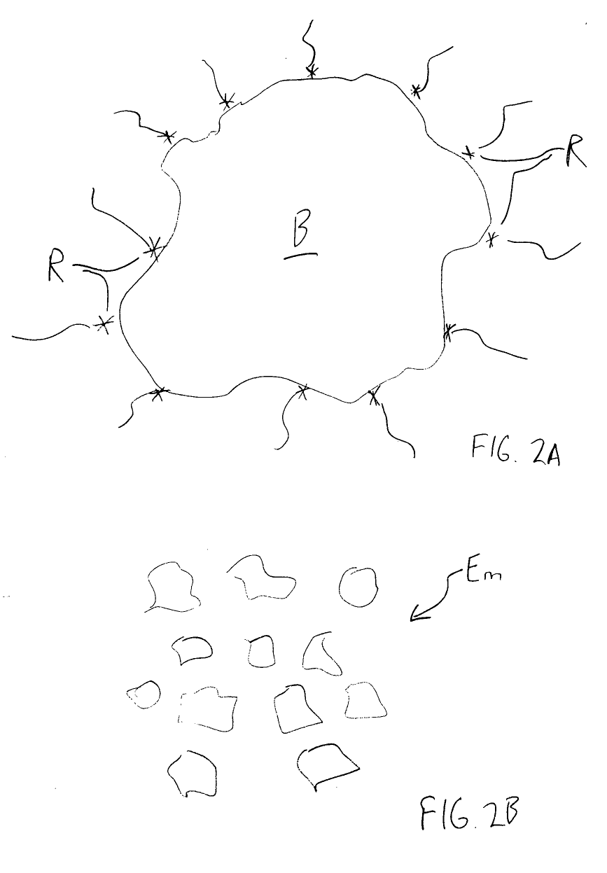

Referring now to FIG. 3, a first embodiment of apparatus in accordance with the present invention is described. Apparatus 10 comprises optical fiber 12 having proximal end 13 and distal end 14. Distal end 14 comprises photocatalyst 16, while proximal end 13 is coupled to energy source 18. Apparatus 10 optionally may comprise radiopaque marker 19, such as a platinum, gold, or iridium marker, near distal end 14 of optical fiber 12 to facilitate proper positioning of apparatus 10 within a patient's body lumen. Energy source 18, e.g. a laser, is adapted to excite or form electron hole pairs within photocatalyst 16. Energy from energy source 18 passes through optical fiber 12 to photocatalyst 16, where it facilitates formation of the radical species in appropriate environments. Energy source 18 may be pulsed in order to control an extent of radical generation and/or diffusion.

Photocatalyst 16 may comprise, for example, a photocatalytic semiconductor, a photosensitizer, or a combination thereof. For the purposes of the present invention, a photocatalyst is defined as a material that is capable of producing a photochemical and/or photophysical alteration in a system, without being consumed by the alteration. A variety of techniques may be used to form photocatalyst 16 on distal end 14 of optical fiber 12, for example, the photocatalyst may be sputter-deposited on the distal end of the optical fiber. Alternatively, the optical fiber may be dipped in a solution of the photocatalyst. As yet another alternative, the photocatalyst may be provided as a liquid, powder, or suspension within an enclosed container at the distal end of the optical fiber. Furtherstill, the photocatalyst may be painted or flame-coated on the surface, or may be deposited via chemical vapor deposition (CVD). Additional deposition techniques will be apparent to those of skill in the art.

When photocatalyst 16 comprises a photocatalytic semiconductor, energy from energy source 18 is adapted to generate electron hole pairs in the photocatalyst. The electron hole pairs generate radical species, such as oxygen-containing radical species, in appropriate environments. Preferred photocatalytic semiconductors 16 include, but are not limited to, TiO₂, SnO₂, and an InTaO₄ compound doped with Ni. Preferred energy sources 18 for use with photocatalytic semiconductors 16 include, but are not limited to, UV and x-ray lasers. Energy source 18 generates energy quanta above the band gap of photocatalytic semiconductor 16.

When photocatalyst 16 comprises a photosensitizer, energy from energy source 18 excites the photosensitizer from a ground state to a singlet excited state. The singlet may decay to an intermediate triplet excited state, which is able to transfer energy to another triplet. Some molecules have a triplet ground state, for example, oxygen or O₂. Thus, energy may be transferred from photosensitizer 16 in the excited triplet state to the triplet ground state molecule, thereby exciting the molecule to a singlet state. A radical-generating reaction may then be achieved with the excited singlet state molecule, for example, a reaction generating oxygen-containing radical species. Molecules, such as thiohydroxamic esters, capable of forming radical species upon exposure to excited photosensitizer 16 will be apparent to those of skill in the art and preferably are provided at distal end 14 of optical fiber 12 when photocatalyst 16 comprises a photosensitizer (not shown). Unlike the liquid photosensitive dyes used in prior art Photodynamic Therapy (“PDT”) techniques, photosensitizers of the present invention are provided in solid form and/or are contained at the distal end of an optical fiber, thereby ensuring that the photosensitizer is not left within the patient post-treatment.

Preferred photosensitizers 16 include, but are not limited to, photofrins, texaphyrins, metallotexaphyrins, porphyrins, hematoporphyrins, chlorins, bacteriochlorins, phthalocyanines and purpurins. Preferred energy sources 18 for use with photosensitizers 16 include, but are not limited to, visible light sources, such as light sources with wavelengths between about 550-850 nm, for example, visible laser light sources, such as Helium Neon (“HeNe”) lasers. Other light sources, including UV light sources, will be apparent. Energy source 18 is capable of exciting photosensitizer 16.

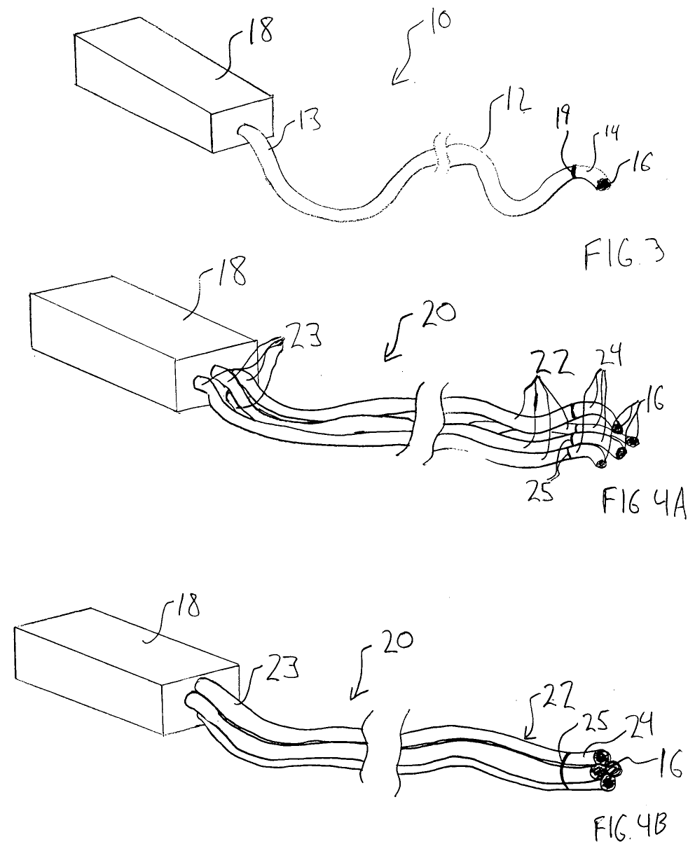

With reference to FIG. 4, a second embodiment of the present invention is described. Apparatus 20 comprises a plurality of optical fibers 22. In FIG. 4, the plurality of fibers illustratively comprises four individual fibers, but any number of fibers may be provided. As with fiber 12 of apparatus 10, each of the individual fibers forming plurality of fibers 22 comprises a proximal end 23 and a distal end 24. Each distal end 24 comprises photocatalyst 16, while each proximal end 23 is coupled to energy source 18. In FIG. 4, the plurality of fibers 22 is coupled to a single energy source 18; however, multiple, potentially diverse, energy sources may be provided, for example, an energy source for each individual fiber. Apparatus 20 optionally may comprise one or more radiopaque markers 25, such as platinum, gold, or iridium markers, near distal ends 24 of optical fibers 22 to facilitate proper positioning of apparatus 20 within a patient's body lumen.

In FIG. 4A, plurality of optical fibers 22 comprises a plurality of discrete optical fibers. In FIG. 4B, plurality of optical fibers 22 comprises a plurality of coupled optical fibers. As will be apparent, a plurality of optical fibers alternatively may be provided that is partially coupled and/or partially discrete.

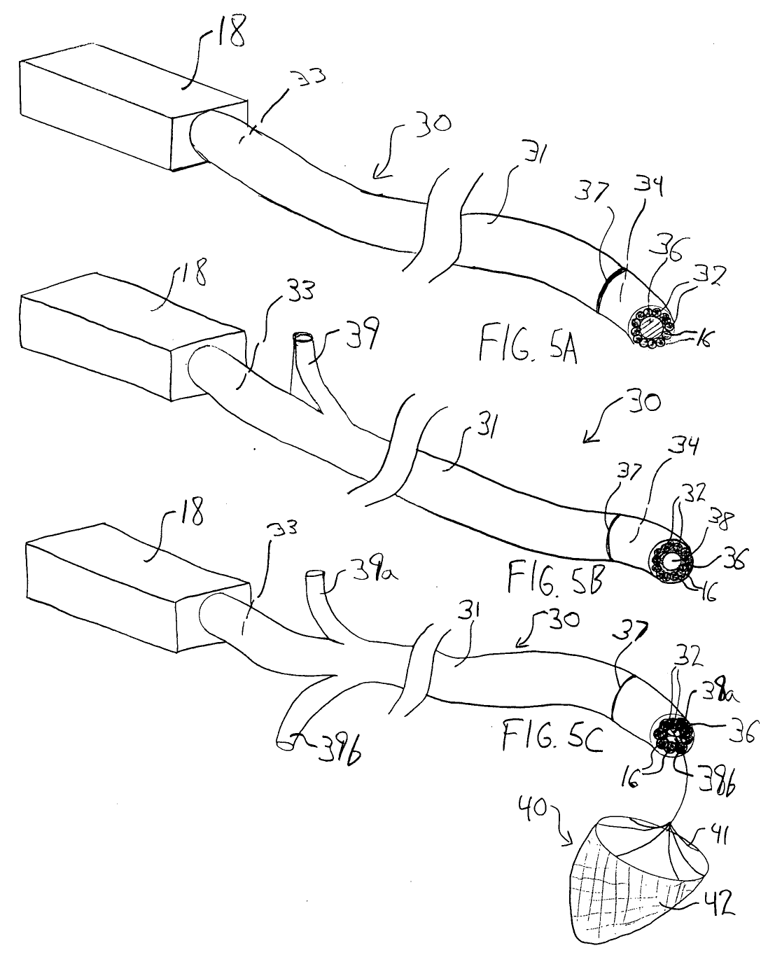

Referring to FIG. 5, a third embodiment of apparatus of the present invention is described. Apparatus 30 comprises a plurality of coupled optical fibers 32 disposed about central shaft 36. Fibers 32 may be formed integrally with shaft 36, for example, via an extrusion process, may be attached to shaft 36 via a secondary joining operation, or may be coupled via optional external sheath 31 disposed coaxially about the fibers. Additional coupling techniques will be apparent to those of skill in the art.

As in the previous embodiment, fibers 32 each comprise a proximal end 33 and a distal end 34. Each distal end 34 comprises photocatalyst 16, while each proximal end 33 is coupled to energy source 18. Plurality of fibers 32 are illustratively coupled to a single energy source 18; however, multiple, potentially diverse, energy sources may be provided. Apparatus 30 optionally may comprise one or more radiopaque markers 37, such as platinum, gold, or iridium markers, near distal ends 34 of optical fibers 32 to facilitate proper positioning of apparatus 30 within a patient's body lumen.

In FIG. 5A, central shaft 36 comprises a solid shaft. Central shaft 36 may be provided such that fibers 32 are spaced with respect to one another and thereby treat a larger surface area of an intraluminal blockage. Additionally, central shaft may facilitate intraluminal advancement of apparatus 30, for example, by increasing the pushability or torqueability of apparatus 30.

In FIG. 5B, central shaft 36 further comprises lumen 38. Lumen 38 may comprise a guide wire lumen, an infusion lumen, or a combination thereof. Lumen 38 proximally terminates at side port 39. As will be apparent to those of skill in the art, lumen 38 may alternatively be provided in a rapid exchange (“RX”) configuration wherein the lumen proximally terminates closer to the distal end of central shaft 36, for example, in a skive through a side wall of the shaft. Rapid exchange catheters are described, for example, in Reexamined U.S. Pat. No. 4,762,129 (1501st Reexamination Certificate), which is incorporated herein by reference.

When using a guide wire, a distal end of the guide wire may be positioned proximate an intraluminal blockage. Apparatus 30 may then be advanced over the guide wire to the vicinity of the blockage. Proper positioning of apparatus 30 may be confirmed, for example, via fluoroscopic imaging of optional radiopaque marker 37. When lumen 38 is used for infusion, a medium may be passed through the lumen, for example, to facilitate generation of radical species and/or to cool the patient's body lumen.

In FIG. 5C, central shaft 36 comprises first lumen 38a and second lumen 38b. First lumen 38a may comprise a guide wire lumen, while second lumen 38b may comprise an infusion lumen. First lumen 38a proximally terminates at first side port 39a, while second lumen 38b proximally terminates at second side port 39b. As will be apparent to those of skill in the art, first lumen 38a may alternatively be provided in a rapid exchange configuration wherein the lumen proximally terminates closer to the distal end of central shaft 38, for example, in a skive through a side wall of the shaft. Additionally, first and second lumens 38 are illustratively shown as a bitumen within central shaft 36; however, lumens 38 may alternatively be provided as coaxial lumens. Furtherstill, additional lumens in excess of two may be provided.

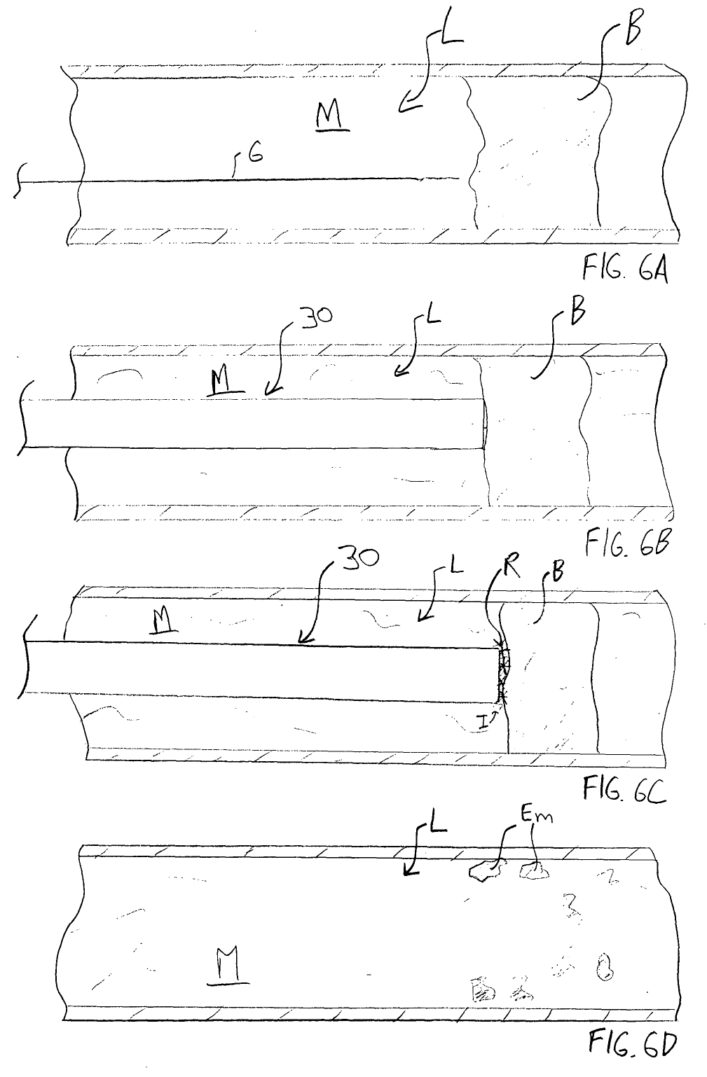

In FIG. 5C, optional embolic protection device 40 is shown. Embolic protection device 40 illustratively comprises expandable filter 41, which is attached to filter sac 42 and is adapted for distal embolic protection. Embolic protection device 40 may be advanced passed an intraluminal blockage in a collapsed delivery configuration, for example, within first lumen 38a. Device 40 may then be expanded to the deployed configuration of FIG. 5C distal of the intraluminal blockage. As radical species break up or dissolve the blockage, as described hereinbelow with respect to FIG. 6, expandable filter 41 and sac 42 of device 40 are adapted to capture potentially harmful emboli formed via dissolution of the blockage.

Preferably, emboli formed during dissolution are smaller than about 100 μm, and even more preferably are smaller than about 60 μm, thereby reducing a risk of harm to the patient from the emboli. Embolic protection device 40 preferably is at least adapted to capture emboli greater than about 100 μm. This may be accomplished, for example, by providing filter sac 42 with pores of about 100 μm or less in cross-section. Pores of about 60-80 μm are preferred, thereby ensuring capture of larger emboli while still allowing passage of intraluminal materials, such as blood cells, therethrough.

Additional expandable filter embolic protection devices are described, for example, in U.S. Pat. No. 6,348,062 to Hopkins et al., which is incorporated herein by reference. As an alternative to expandable distal protection devices, embolic protection device 40 may comprise any known embolic protection device, including, for example, a proximal protection device, such as a suction catheter. Suction optionally may be drawn through first or second lumen 38 of apparatus 30. Additional proximal suction embolic protection devices are described, for example, in U.S. Pat. No. 6,295,989 to Connors, III, which is incorporated herein by reference. Other embolic protection devices, per se known, will be apparent to those of skill in the art.

Referring now to FIG. 6, in conjunction with FIGS. 1, 2 and 5C, a method of using the apparatus of FIG. 5C is described. In FIG. 6a, body lumen L, for example, a blood vessel, comprises intraluminal blockage B, such as a blood clot, thrombosis, or other intraluminal occlusion. Medium M is disposed within lumen L. Medium M preferably is capable of generating radical species in the presence of electron hole pairs or excited molecules, for example, an oxygen-containing medium, such as blood, water, oxygen, air, saline or a combination thereof. If an appropriate medium is not naturally occurring within lumen L in the vicinity of blockage B, a clinical practitioner optionally may provide it, for example, via a guiding or infusion catheter, or via apparatus of the present invention. In FIG. 6a, optional guide wire G has been advanced within lumen L proximate blockage B, for example, using well-known percutaneous techniques. The guide wire may alternatively be advanced within or past the blockage.

In FIG. 6B, apparatus 30 of FIG. 5C has been advanced over optional guide wire G, for example, by advancing the distal end of first lumen 38a over the proximal end of guide wire G. Photocatalyst 16, disposed on distal ends 34 of the plurality of coupled optical fibers 32, is positioned in close proximity or contact with intraluminal blockage B. Proper positioning may be achieved, for example, via fluoroscopic imaging of optional radiopaque marker 37.

In FIG. 6C, energy source 18 is activated and transmits energy through optical fibers 32 to photocatalyst 16. The energy generates radical species at the interface of photocatalyst 16 with medium M. As discussed previously with respect to FIGS. 1A and 1B, when photocatalyst 16 comprises a photocatalytic semiconductor, electron hole pairs are generated within the photocatalytic semiconductor because energy source 18 excites photocatalyst 16 with energy above the band gap of the semiconductor. As discussed previously with respect to FIG. 1C, when photocatalyst 16 comprises a photosensitizer, incident light excites the photosensitizer in a manner capable of generating radical species upon contact with appropriate molecules, for example, oxygen molecules or thiohydroxamic esters, which are preferably incorporated into distal ends 34 of fibers 32.

It is expected that radical species R formed at the interface of medium M and photocatalyst 16 typically will be capable of traveling on the order of 100 nm. It is further expected that radical species R will be transferred from the interface of medium M and photocatalyst 16 to the interface of medium M and intraluminal blockage B along a substantially shortest distance path. Thus, only the blockage in close proximity to photocatalyst 16 will come into contact with radical species R. Portions of the body lumen L that are not contacted by the radical species are not expected to oxidize, dissolve, break up, etc., thereby reducing a risk of damage to other intraluminal structures.

As seen in FIG. 6D, and discussed previously with respect to FIG. 2, the radical species locally oxidize or transfer energy to blockage B, which breaks up or dissolves the blockage into smaller pieces or emboli Em. It should be noted that oxidation and/or energy transfer to blockage B may be possible with excited singlet or triplet state molecules, in addition to radical species. As discussed previously, emboli Em are preferably smaller than about 100 μm, and even more preferably smaller than about 60 μm, in order to reduce a risk of harm to the patient from the emboli. Optionally, embolic protection may be provided to capture larger emboli Em, for example, embolic protection device 40 of FIG. 5C or suction drawn through second lumen 38b of apparatus 30. Once blockage B has been broken up or dissolved, apparatus 30, as well as optional guide wire G, may be removed from body lumen L, thereby treating blockage B without leaving foreign materials resident in lumen L post-treatment.

Prior to, during, or after activation of energy source 18, optional infusion medium I may be delivered within body lumen L in the vicinity of blockage B. Infusion medium I may be delivered through a guiding catheter, an infusion catheter, or through second lumen 38b of apparatus 30 of FIG. 5C. Infusion medium I may comprise, for example, oxygen, air, water, saline or a combination thereof, and may be provided to cool lumen L and/or blockage B during treatment. Additionally or alternatively, infusion medium I may enhance or facilitate formation of radical species R.

Lumen L of FIG. 6 may comprise any body lumen experiencing a blockage. These include, but are not limited to, blood vessels, heart valves, biliary ducts, the urethra or prostate, the bladder, the stomach, the throat, fallopian tubes, etc. Additional lumens will be apparent to those of skill in the art.

Energy source 18 preferably comprises an energy source having fixed operational parameters suited for use in a specific clinical indication and/or with a specific embodiment of the present invention. It is expected that providing fixed parameters will simplify the procedure for a medical practitioner, while reducing time and associated costs. Energy source 18 alternatively may be provided with adjustable parameters to increase its applicability to more diverse clinical indications and/or embodiments of the present invention.

When photocatalyst 16 comprises a photocatalytic semiconductor, the band gap energy of the photocatalytic semiconductor is dictated by:

E=hν (6)

where h is Plank's constant and equals 1.603×10⁻¹⁹, and E is the band gap energy of photocatalytic semiconductor 16. Since ν is the frequency of energy from energy source 18, and is related to the wavelength λ of the energy by:

ν=C/λ (7)

where C equals the speed of light, the excitation energy of can be specified such that it is above the band gap energy E of photocatalytic semiconductor 16 by choosing an energy source 18 capable of generating energy of appropriate wavelength. As an example, when photocatalyst 16 comprises TiO2, the band gap energy is 3.2 eV, which may be generated by the wavelength of light produced, for example, with either a UV or x-ray energy source 18.

Although the equations above are believed to describe the band gap energy of a photocatalytic semiconductor, the present invention is primarily concerned with the end result, i.e. treatment of intraluminal blockages. Thus, these equations are provided only for the benefit of the reader and should in no way be construed as limiting.

A significant advantage of the present invention, as compared to prior art Photodynamic Therapy techniques, is that PDT requires introduction and local accumulation of a photosensitive dye or drug over a period of time at a target site. Such localized accumulation is difficult or impractical in many body lumens where fluids are flowing, such as in blood vessels containing blood. Conversely, the present invention only requires that photocatalyst 16 be exposed to an appropriate medium, which need not be localized nor allowed to locally accumulate over a period of time. The medium may be chosen such that a risk of harm to the patient due to the medium is negligible. Such media may include, for example, water, oxygen, air, saline and combinations thereof. Furthermore, the medium preferably is naturally occurring at the treatment site. For example, when treating an intravascular blockage, the medium may comprise blood. In such cases, no foreign material is left in the patient post-treatment, since the photocatalyst is disposed at the distal end of an optical fiber that is removed from the patient post-treatment.

As compared to prior art photoacoustic emulsification techniques, the present invention advantageously requires relatively low energy concentrations and does not require formation of a concussive wave.

While preferred illustrative embodiments of the invention are described hereinabove, it will be apparent to one skilled in the art that various changes and modifications may be made therein without departing from the invention. For example, apparatus may be provided comprising a plurality of optical fibers, each having a different photocatalyst at its distal end. When the photocatalysts comprise multiple photocatalytic semiconductors, each may comprise a different band gap potential. When they comprise multiple photosensitizers, each may comprise a different excitation energy. A mixture of photocatalytic semiconductors and photosensitizers may also be provided. In such embodiments, multiple energy sources may be provided, each capable of generating energy at a different excitation level. Alternatively, a tuneable energy source may be provided. The appended claims are intended to cover all such changes and modifications that fall within the true spirit and scope of the invention. Additionally, it should be understood that the previously described Figures are schematic and are not necessarily drawn to scale.

What is claimed is:

1. Apparatus for treating intraluminal blockages, the apparatus comprising:
an optical fiber having proximal and distal ends;
a photocatalyst coupled to the distal end of the optical fiber; and
an energy source coupled to the proximal end of the optical fiber,
wherein the energy source is adapted to excite the photocatalyst and generate radical species in the presence of an appropriate medium.

2. The apparatus of claim 1, wherein the intraluminal blockage comprises a blockage chosen from the group consisting of blood clots, thrombosis, stones, plaque, and intraluminal occlusions.

3. The apparatus of claim 1, wherein the photocatalyst comprises a photocatalytic semiconductor adapted to generate electron hole pairs upon excitation by the energy source above a band gap of the photocatalytic semiconductor, and wherein the electron hole pairs generate the radical species in the presence of the appropriate medium.

4. The apparatus of claim 1, wherein the medium is adapted to transport the radical species from the photocatalyst to the intraluminal blockage.

5. The apparatus of claim 1, wherein the radical species are adapted to locally oxidize or transfer energy to the intraluminal blockage at points where the radical species contact the blockage.

6. The apparatus of claim 1, wherein the photocatalyst is chosen from the group consisting of photocatalytic semiconductors, TiO₂, SnO₂, compounds of InTaO₄ doped with Ni, photosensitizers, photofrins, texaphyrins, metallotexaphyrins, porphyrins, hematoporphyrins, chlorins, bacteriochlorins, phthalocyanines, purpurins, and combinations thereof.

7. The apparatus of claim 1, wherein the energy source is chosen from the group consisting of pulsed sources, visible light sources, UV sources, x-ray sources, visible light lasers, HeNe lasers, UV lasers, x-ray lasers, pulsed lasers, and combinations thereof.

8. The apparatus of claim 1, wherein the medium is chosen from the group consisting of blood, oxygen, air, water, saline and combinations thereof.

9. The apparatus of claim 1 further comprising one or more additional optical fibers having proximal and distal ends, the photocatalyst coupled to the distal ends and the energy source coupled to the proximal ends.

10. The apparatus of claim 9 further comprising a central shaft, the optical fibers disposed about the central shaft.

11. The apparatus of claim 1 further comprising a radiopaque marker disposed near the distal end of the optical fiber.

12. The apparatus of claim 1 further comprising an embolic protection device.

13. A method for treating an intraluminal blockage, the method comprising:
removably disposing a photocatalyst in close proximity or contact to the blockage;
exciting the photocatalyst;
generating radical species with the excited photocatalyst;
transferring the radical species to the blockage; and
locally oxidizing or transferring energy to the blockage at points where the radical species contact the blockage, thereby disrupting the blockage.

14. The method of claim 13, wherein removably disposing the photocatalyst comprises removably disposing a photocatalytic semiconductor, and wherein exciting the photocatalyst comprises forming electron hole pairs in or on the photocatalytic semiconductor by exciting the photocatalytic semiconductor above its band gap.

15. The method of claim 14, wherein generating radical species with the excited photocatalyst comprises generating radical species by contacting the electron hole pairs with an appropriate medium in communication with the photocatalytic semiconductor.

16. The method of claim 13, wherein removably disposing the photocatalyst comprises removably disposing a photosensitizer, and wherein exciting the photocatalyst comprises exciting the photosensitizer.

17. The method of claim 13, wherein removably disposing a photocatalyst comprises providing the photocatalyst on the distal end of an optical fiber that is removably disposed in close proximity or contact to the blockage, and wherein exciting the photocatalyst comprises transferring energy to the photocatalyst through the fiber.

18. The method of claim 13 further comprising delivering an infusion medium proximate the intraluminal blockage.

19. The method of claim 13 further comprising capturing emboli formed while disrupting the blockage.

20. Apparatus for treating an intraluminal blockage, the apparatus comprising:
a photocatalyst adapted for removable disposal proximate the blockage; and
an energy source adapted to excite the photocatalyst,
wherein excitation of the photocatalyst generates radical species in appropriate media,
wherein the radical species are adapted to locally oxidize or transfer energy to the intraluminal blockage, thereby disrupting the blockage.
